(19) United States
(12) Patent Application Publication
(10) Pub. No.: US 2006/0173274 A1
(43) Pub. Date: Aug. 3, 2006

(54) Functional magnetic resonance imaging guided transcranial magnetic stimulation deception inhibitor

(76) Inventors: Mark S. George, Sullivan's Island, SC (US); Frank A. Kozel, Southlake, TX (US); Daryl E. Bohning, Warren, CT (US)

Correspondence Address: NEEDLE & ROSENBERG, P.C., SUITE 1000, 999 PEACHTREE STREET, ATLANTA, GA, 30309-3915, US

(21) Appl. No.: 10/521,373

(22) PCT Filed: Jul. 11, 2003
(86) PCT No.: PCT/US03/21660

§ 371 Date: Jan. 3, 2006

Related U.S. Application Data
(60) Provisional application No. 60/396,054, filed on Jul. 15, 2002.

(51) Int. Cl. A61B 5/05

(52) U.S. Cl. 600/409

(57) ABSTRACT

Functional brain imaging is used to determine the regions of a brain an individual uses to lie or deceive, and then the TMS is applied to that region of the brain while the individual, is, e.g., attempting to respond to a question. If the person is attempting to deceive, TMS will temporarily inhibit operation of this part of the brain during this attempted deception, and the individual will be unable to deceive.

### CROSS REFERENCE TO RELATED APPLICATION

This application claims the benefit of priority from U.S. Provisional Patent Application No. 60/396,054 filed Jul. 15, 2002, herein incorporated by reference.

### BACKGROUND

Deception, defined herein as the purposeful misleading of another, is common. People often mislead others to gain an advantage or to protect themselves or others. There are many military, legal, political, and industrial settings where society could benefit from an accurate method for detecting deception. A variety of technologies and approaches have been developed in the area of deception detection.

Presently, there are a number of lie-detection testing techniques that use polygraph devices. All of the devices examine the peripheral autonomic response to relevant versus irrelevant questions. For example, present day polygraph devices record electro-dermal skin conductance in addition to changes in blood pressure, respiration and peripheral vasomotor activity. Whenever a greater autonomic response to the relevant questions versus the irrelevant or control questions is recorded, this data is interpreted as an attempt to deceive by the individual that is being tested.

Polygraph devices have several significant limitations, including the ability of test subjects to develop countermeasures to the techniques that are utilized to detect deception. An additional problem with polygraph devices is that they do not posses the capability to test for a subject's deception but rather measure non-specific peripheral changes in the arousal of the test subject. The substantive predictive value of the polygraph has been found to be poor in many screening and investigative situations, and scientific evidence regarding the polygraph's validity is significantly lacking. Despite these and other shortcomings, the polygraph continues to be used widely in job screening and criminal investigations.

Various other techniques have been investigated to predict deception; all of which use measurement of peripheral arousal responses. These techniques include measuring papillary size response to visual stimuli that are mock crime scene related, using voice analysis, facial and hand movement cues to identify subjects who are lying or being truthful, observing verbal cues to detect a true life tale versus a fabricated one, attempting to detect deception in and out of hypnosis, and using high-definition thermal imaging techniques to detect periorbital changes in people trying to deceive. One of the few methods to measure actual brain activity to detect deception involves examining the amplitude of the P300 component of event-related brain potentials. Even if it proves effective, however, this technique has limited utility since it is only applicable when attempting to detect guilty knowledge.

There is a deficiency of knowledge in regard to the neurobiological or brain basis of the polygraph. This is shortcoming has lead to advances in functional brain imaging. Researchers, using functional MRI and Positron Emission Tomography (PET) have successfully delineated the brain changes involved in response inhibition (e.g. Go/No-Go tasks), divided attention, anxiety, emotion-related learning with reward and punishment, and differentiating components of cognitive breakthrough.

Very limited techniques have been developed to actually inhibit deception rather than merely detect it. One such approach is the use of amobarbital sometimes referred to as “truth serum.” This is a pharmacologically based method for inhibiting deception. The exact regions and mechanisms of how amobarbital does this is not known; however, amobarbital provides a mechanism for effecting the specific behavioral change of inhibiting deception.

Transcranial magnetic stimulation (TMS) techniques have been developed over the years to achieve a variety of motor and behavioral changes in subject individuals. Such techniques have heretofore not been applied to the inhibition of deception. For over a century, it has been recognized that electricity and magnetism are interdependent (Maxwell's equations)(Bohning, 2000). Passing current through a coil of wire generates a magnetic field perpendicular to the current flow in the coil. If a conducting medium, such as the brain, is adjacent to the magnetic field, current will be induced in the conducting medium. The flow of the induced current will be parallel, but opposite in direction, to the current in the coil (Cohen et al., 1990; Brasil-Neto et al., 1992; Saypol et al., 1991; Roth et al., 1991). Thus, TMS has been referred to as “electrodeless” electrical stimulation to emphasize that the magnetic field acts as the medium between electricity in the coil and induced electrical currents in the brain.

TMS involves placing an electromagnetic coil on the scalp. Subjects are awake and alert. There is some discomfort, in proportion to the muscles that are under the coil, and to the intensity and frequency of stimulation. Subjects usually notice no adverse effects except for occasional mild headache and discomfort at the site of the stimulation. High intensity current is rapidly turned on and off in the coil through the discharge of capacitors. This produces a time-varying magnetic field that lasts for about 100-200 microseconds. The magnetic field typically has a strength of about 2 Tesla (or 40,000 times the earth's magnetic field, or about the same intensity as the static magnetic field used in clinical MRI). The proximity of the brain to the time-varying magnetic field results in current flow in neural tissue. The technological advances made in the last 15 years led to the development of magnetic stimulators that produce sufficient current in brain to result in neuronal depolarization.

A striking effect of TMS occurs when one places the coil on the scalp over primary motor cortex. A single TMS pulse of sufficient intensity causes involuntary movement. The magnetic field intensity needed to produce motor movement varies considerably across individuals, and is known as the motor threshold (Kozel et al., 2000; Pridmore et al., 1998). Placing the coil over different areas of the motor cortex causes contralateral movement in different distal muscles, corresponding to the well-known homunculus. TMS can be used to map the representation of body parts in the motor cortex on an individual basis. Subjectively, this stimulation feels much like a tendon reflex movement. Thus, a TMS pulse produces a powerful but brief magnetic field which passes through the skin, soft tissue and skull, and induces electrical current in neurons, causing depolarization which then has behavioral effects (body movement).

Single TMS over motor cortex can produce simple movements. Over primary visual cortex, TMS can produce the perception of flashes of light or phosphenes (Amassian et al., 1995). To date, these are the ‘positive’ behavioral effects of single pulse TMS. Other immediate behavioral effects are generally disruptive. Interference with, and perhaps augmentation of, information processing and behavior is especially likely when TMS pulses are delivered rapidly and repetitively. Repeated rhythmic TMS is called repetitive TMS (rTMS). If the stimulation occurs faster than once per second (1 Hz) it is modified as fast rTMS.

The ability of TMS to effect cognitive/behavioral or physical alteration has been demonstrated in several areas. Topper et al have shown that stimulation over temporal lobe facilitates or improves picture naming (Topper et al., 1998). Grafman et al. have recently shown that stimulation over the prefrontal cortex, and not sham stimulation, improves analogous reasoning (Boroojerdi et al., 2001). The same NIH group has shown that 1 Hz TMS for 10 minutes can transiently suppress motor cortex or visual cortex activity, for up to 20 minutes following stimulation.

According to the present invention, non-pharmacological systems and methods to inhibit deception involve the application of TMS to brains regions determined to be related to deception as determined through one or more functional brain mapping techniques.

### SUMMARY

According to exemplary embodiments, functional brain imaging is used to determine the regions an individual uses to lie or deceive, and then the TMS is applied to that region of the brain while the individual is attempting to respond to a question. If the person is attempting to deceive, TMS will temporarily inhibit operation of this part of the brain during this attempted deception, and the individual will be unable to deceive.

According to an exemplary embodiment, a TMS deception-inhibiting device is guided by functional brain imaging. In one embodiment, functional brain mapping using fMRI is used in conjunction with specific methods of placing the TMS device over the identified regions of the brain. Embodiments of the present invention, however, are designed to extend beyond these specific technical methods, and cover as well any method of functional brain imaging (including but not limited to PET, SPECT, qEEG, MEG), as well as any method for positioning the TMS device, within or outside of the actual scanner. Moreover, the ability of TMS to produce focal lesions is not specific to any one form of TMS device (figure eight, round, etc), or any one TMS manufacturer.

In one embodiment, fMRI is used to determine the brain region or regions that show activation while the person is deceiving. Once this area is identified using fMRI (or other brain imaging methods), TMS is applied over this region to temporarily inhibit (or turn off) the ability to deceive. This inhibition is unique to deception and does not interfere with truthful responses. TMS applied to the regions identified as being important in deceiving make the subject unable to give deceitful answers.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention.

### BRIEF DESCRIPTION OF DRAWINGS

FIG. 1A depicts a subject individual being positioned for functional brain imaging using an MRI scanner.

FIGS. 1B-1F illustrate details of a skin conductance monitoring system that may be used in conjunction with an MRI scanner.

FIG. 2 depicts a subject individual with a TMS system including a translational/positioning system.

FIGS. 3A-B are sample screens shown to test subjects during questioning for determining brain regions important for deception.

FIG. 4 depicts brain imaging derived from fMRI scanning during periods of deception.

FIG. 5 depicts brain imaging derived from fMRI scanning correlated with EDA measures during periods of deception.

### DETAILED DESCRIPTION

One or more preferred embodiments of the invention are now described in detail. Referring to the drawings, like numbers indicate like parts throughout the views. As used in the description herein, the meaning of “a,” “an,” and “the” includes plural reference unless the context clearly dictates otherwise. Also, as used in the description herein, the meaning of “in” includes “in” and “on” unless the context clearly dictates otherwise. Finally, as used in the description herein and throughout the claims that follow, the meanings of “and” and “or” include both the conjunctive and disjunctive and may be used interchangeably unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The systems and methods according to exemplary embodiments can involve use of a variety of equipment depending upon the particular embodiment. Therefore, the various specific equipment described herein is to be taken as exemplary only; those skilled in the art will readily appreciate that other equipment providing similar functionality can be used instead of or in addition to the specific exemplary equipment described herein.

Functional Brain Imaging

According to some embodiments, functional brain imaging is applied to a subject to determine brain regions that experience significant activation during periods where the subject is making deceptive statements. In some embodiments, the functional brain mapping occurs solely as a calibration phase for determining relevant brain regions for use during an inhibition phase. In other embodiments, the functional brain imaging occurs during both a calibration phase and an inhibition phase. In such embodiments, real-time functional brain imaging data is initially gathered during the calibration phase and used to initiate the inhibition phase; further real-time data accumulated during the inhibition phase is then used as feedback to further tune the calibration phase data and enhance the ability to inhibit deception. In yet further embodiments, no calibration phase is required; rather, real-time functional brain imaging data is accumulated during questioning of the subject. This imaging data is refined during the questioning so that the ability to inhibit deception improves over the course of questioning. Any suitable functional brain imaging technique can be used including without limitation fMRI, PET, SPECT, qEEG and MEG.

In some embodiments, real-time blood oxygen level dependent (BOLD) functional MRI (fMRI) analysis offers one approach to functional brain imaging. This approach enables the rapid interpretation of functional imaging results, even while the subject is still in the scanner performing the task. This method is very useful in the pre-surgical mapping of language areas within the brain. In its current implementations, fMRI appears sensitive enough to detect brain regions involved in many of the cognitive and emotional tasks involved in deception. This technology and recently developed expertise that allows for the recordation of electro-dermal activity (an important component used in current polygraph devices) during fMRI scanning, has led to vast improvements in the field of deception detection. Thus, the field of functional brain imaging has rapidly developed to the point where it is theoretically possible to use scanning to detect people who are trying to deceive, and further, allow for the coupling of this technology with other measures like the polygraph. This approach is described in greater detail in Applicant's U.S. Provisional Patent Application Ser. No. 60/341,297, filed Dec. 13, 2001, entitled “System and Method of Detecting Deception by fMRI” and the corresponding International Application No. PCT/US02/40142 filed Dec. 13, 2002. The content of both of these applications is hereby incorporated by this reference herein for all purposes.

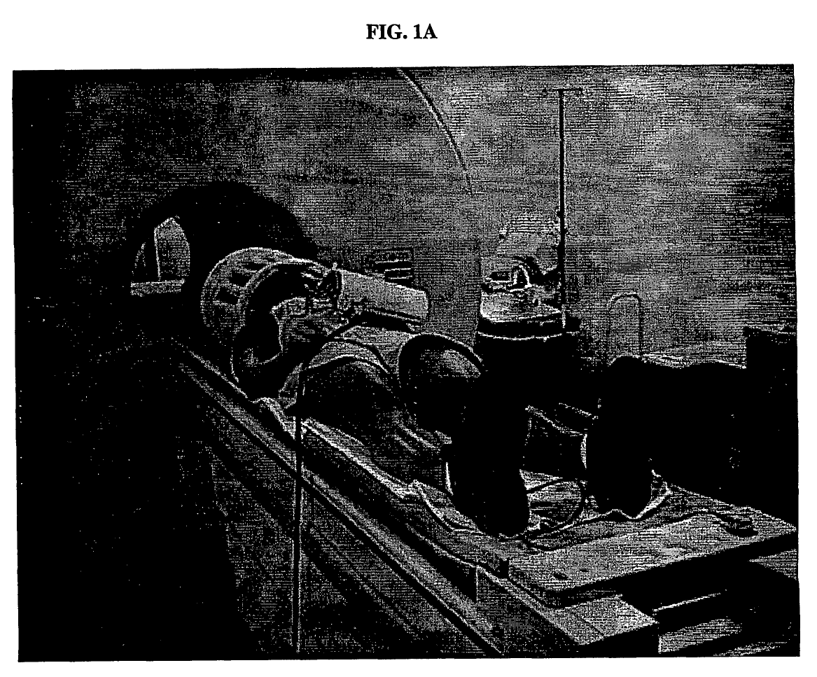

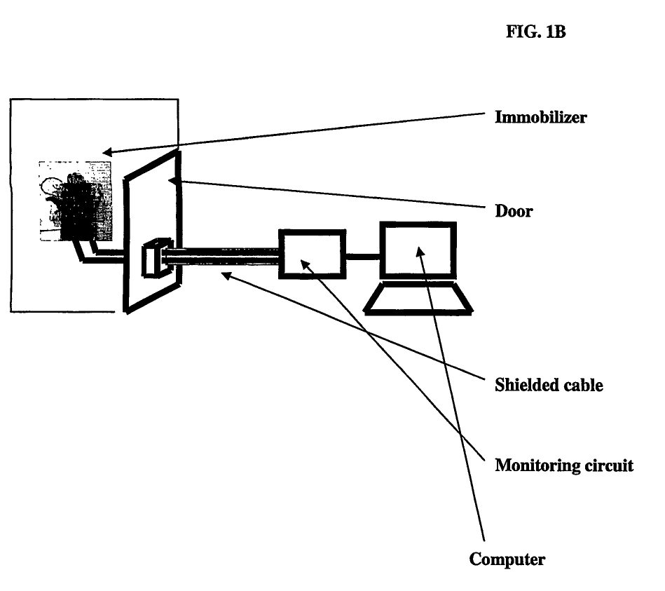

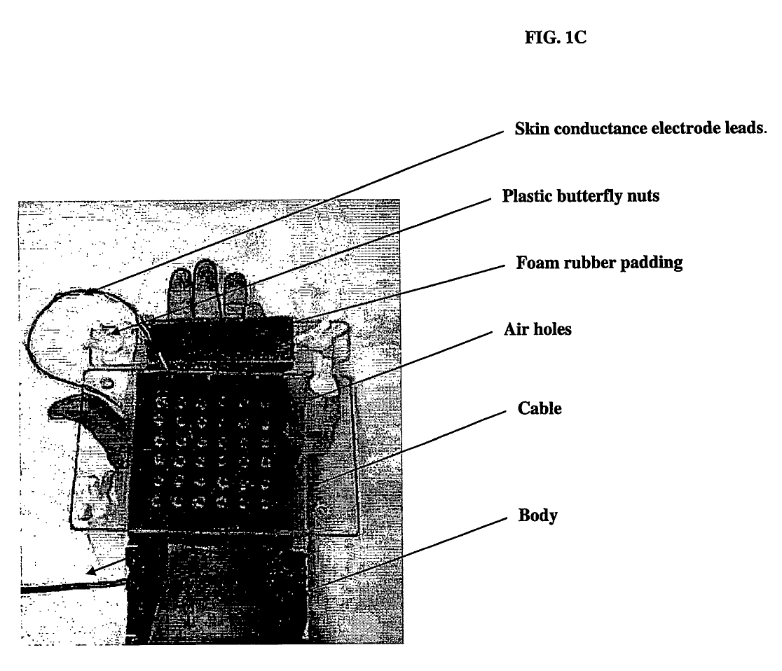

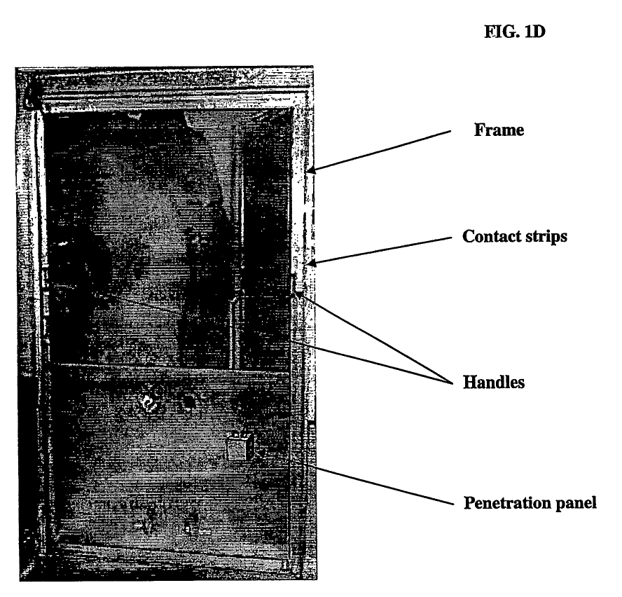

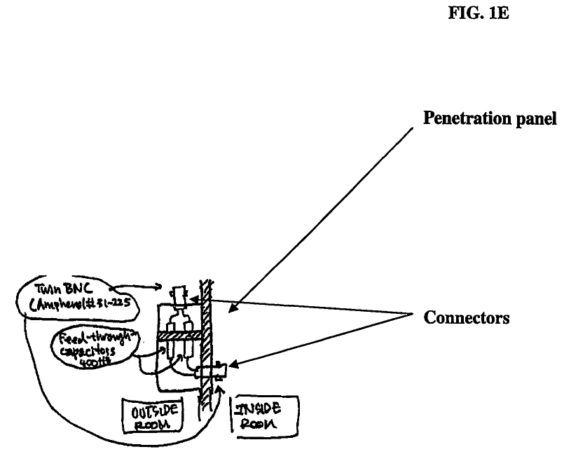

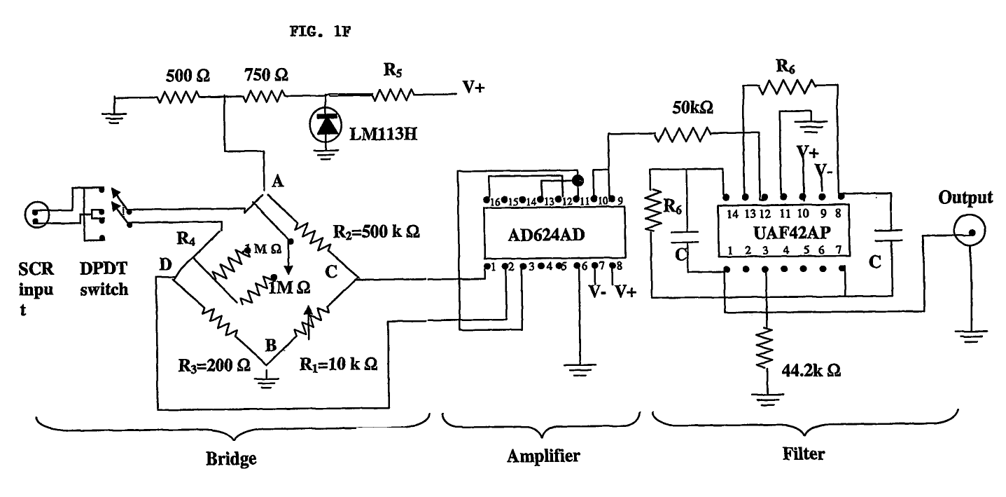

In one such embodiment, the subject is next placed in a fMRI scanner such as 1.5 Tesla Philips or Picker Edge 1.5T scanner and a structural picture of the brain is acquired as depicted in FIG. 1A. Next, a series of questions for which the questioner knows the answer are asked in which the person makes either truthful or deceptive answers.

The blood flow pattern recorded during truthful statements is subtracted from the blood flow pattern recorded during deceptive statements. Previous research has found significant activation in the right orbitofrontal and/or cingulate regions of the brain during periods of deception; however, other brain regions can be of significance as well. Using real-time functional image analysis, the area(s) of activation in the brain for that person during deception is identified and targeted with the TMS.

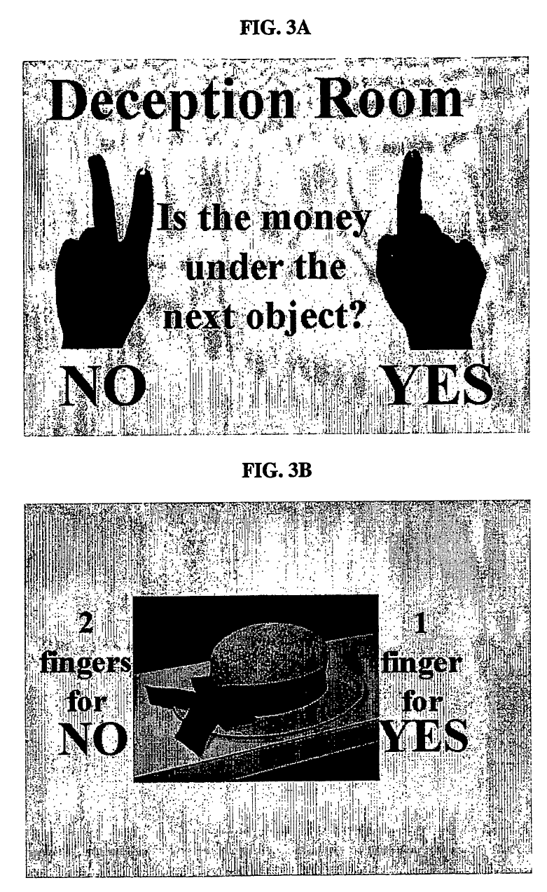

A test of one such embodiment was performed using 8 male test subjects. While the BOLD fMRI scans were being acquired, a modified Control Question Test paradigm was utilized in which the subjects would give both truthful and deceitful answers about the location of the money. Through video goggles connected to a computer, the subjects were shown prompt screens and then pictures of the objects in the rooms where the money had been hidden as depicted in FIGS. 3A-B. If the subjects first looked in the TRUTH room, then they were shown only the TRUTH room objects first and then the DECEPTION room objects and vice versa if subjects were first shown the DECEPTION room. There were five objects in each room (ten unique objects in all), and the objects were each shown one time in a block for a total of four blocks per room.

| Truth Room | | Deception Room | |
|---|---|---|---|
| Coffee Pot | Prompt | *Hat | Prompt |
| | Image | | Image |
| *Shoe | Prompt | Mouse | Prompt |
| | Image | | Image |
| Cooler | Prompt | Santa | Prompt |
| | Image | | Image |
| Truck | Prompt | Bowl | Prompt |
| | Image | | Image |
| Plate | Prompt | Telepone | Prompt |
| | Image | | Image |

| | |
|---|---|
| Block of images repeated three | Block of images repeated three |
| times (total 4 blocks) with order | times (total 4 blocks) with order |
| within each block randomly | within each block randomly |
| changed | changed |

The order of the objects was randomized within each block. The items with an asterisk “*” were hiding the fifty dollar bill in the respective rooms. The order of room/image presentation was randomized. Before the picture of the first object and between the pictures of every object in the room, a PROMPT screen was displayed that reminded the subjects of the instructions. The object and the PROMPT were each displayed for 10.2 seconds. Subjects were instructed to raise either one (yes) or two (no) fingers to answer the question of whether the money was hidden under an object as soon as the object was visually displayed in the goggles. This was monitored and recorded by an observer (LR).

For the TRUTH room, subjects were instructed to accurately report the location of the money by holding up the right index finger (one finger) when they were shown an object under which the money had been hidden. This would be the “control” with which the deceptive answers would be compared. This controls for the potential confounds of brain changes associated with simply seeing an object that had money under it. They were instructed to raise the right index and middle finger (two fingers) when shown an object under which the money was not hidden.

For the DECEPTION room, they were to instructed to choose an object that did not have money hidden under it and respond affirmatively (right index finger), in effect creating a positive lie about the money location. They were also asked to respond negatively (right index and middle finger) to all other objects shown. They were thus consistently lying when the object with money hidden under it was shown (a hat). The money was hidden under the same object for all subjects. Subjects were told that a blinded investigator (to order of rooms visited and location of the money) would attempt to determine when they were lying by observing their behavior in the scanner through the control room window. If the subjects accurately reported the position of the money in the TRUTH room, then they would receive 50 dollars. If the subjects “successfully” lied without being detected for the DECEPTION room, then they would receive an additional 50 dollars. In fact, all subjects were paid the full 100 dollars. This instruction was included in order to increase the motivation and the anxiety during deception.

MRI images can be acquired using a Picker Edge 1.5T MRI scanner equipped with an actively shielded magnet and high performance whole-body gradients (27 mT/m, 72 T/m-sec). A 15-slice TE20 structural scan can be obtained to evaluate for any structural pathology. The Blood Oxygen Level Dependent (BOLD) fMRI can consist of 15 coplanar transverse slices (8.0 mm thick/0 mm gap) covering the entire brain and positioned 90 degrees to the Anterior Commissure-Posterior Commissure line using a sagittal scout image. Each fMRI volume can consist of BOLD weighted transverse scans and used an asymmetric-spin gradient echo, echo-planar (EPI) fMRI sequence (tip angle=90° to the Anterior Commissure-Posterior Commissure line; TE 45.0 ms; TR 3000 ms; fifteen 8 mm thick/0 mm gap transverse slices; FOV 300×300 mm; in-plane resolution 2.109×2.109 mm; through-plane resolution 8 mm; frequency selective fat suppression). Given these parameters for the fMRI, a set of fifteen 8 mm thick/0 mm gap transverse slices covering the entire brain can be obtained every 3 seconds.

Functional MRI Analysis

The data were analyzed with MEDx 3.3/SPM96 on Sun workstations using the Talairach and Tournoux brain template throughout. Initially, the MEDx motion detection function was performed using the center of intensity weighting method. Any motion greater than 2.0 mm would have been corrected using the MEDx 3.3 motion correction function (no subjects required motion correction). Next, individual volumes were spatially normalized into Talairach space utilizing the SPM Module 96 in MEDx 3.3. Algorithm parameters included Basic functions and smoothing x=4, y=5, z=1, iteration=2, smoothing=8.0, deformation=0.2, the SPM template corresponding to the original Talairach and Tournoux adas31 and output voxel size 4×4×4 mm. Using the SPM module again, spatial smoothing was performed using 8×8×8 mm gaussian kernel. Intensity normalization was performed which first created a with-in-the-brain mask that only included voxels if they had intensity greater than 35% the maximum of each image volume for all time points and then scaled the remaining non-zero voxels in each volume in the time series to a mean value of 1000. We then performed high pass temporal filtering which filtered out patterns greater than twice the cycle length of 204 seconds. Due to the SPM module performing another intensity mask during the upcoming SPM statistics step, a .tcl script was written to add 100 to all voxels outside the brain. When the SPM statistics was run, this ensured that no voxels we previously defined as within brain would be eliminated from the analysis but that voxels we previously defined as outside the brain would be eliminated.

Using the SPM module on MEDx 3.3, statistical analysis with a delayed boxcar design without temporal filtering was performed. The epochs were grouped as Lie (the time period when individuals gave a false answer—both indicating that the object did not conceal money when it did {4 epochs} and indicating the object concealed money when it did not {4 epochs}), Lprompt (time period prompt image displayed just prior to each Lie {8 epochs}), True1 (time period subjects answered truthfully the location of the money {4 epochs} and 4 truthful answers that the money was not under an object—temporally surrounding deceptive answers {4 epochs}), Prompt1 (time period prompt displayed immediately preceding True1 epochs), True (time period of all remaining truthful answers {24 epochs}), and Prompt (time period of prompt immediately preceding True epochs{24}). Using these epochs, Lie minus True1 and True1 minus Lie was computed with no threshold (p=0.05 and uncorrected k (cluster size)=1). The individual unthresholded images were used to obtain a group and individual activation profiles.

To calculate a GROUP result, for all individuals, the image calculator in MEDx 3.3 was used to compute unthresholded Lie minus True1 z-maps containing both positive and negative z-scores. Thus, we used the image calculator to obtain the result of (Lie minus True1) minus (True1 minus Lie) z-maps for each subject. Once this was obtained for all individuals, they were summed and then divided by the square root of eight to create the group fixed effects analysis unthresholded z-map. The resulting image was then analyzed with MEDx 3.3 cluster detection with a minimum of z=1.645 and spatial extent threshold of 0.05. A low statistical threshold was chosen since our paradigm could only have a limited number of epochs of Lie. In addition, although we were directly testing our hypothesized regions, we were interested in analyzing the whole brain since we had no previous neuroimaging studies to focus our analysis. The resulting values were used to determine local maxima and visually present the significant clusters. The Talairach Daemon interface in MEDx 3.3 was used to identify locations of the local maxima. In addition, the Talairach atlas1 was used to confirm the location of the significant clusters. The Johns Hopkins University BRAID imaging database at URL http://braid.radjhu.edu/index atlases.html determined the Damasio Talairach space definition of orbitofrontal cortex.

For the INDIVDUAL analysis, in a similar fashion to the group analysis, the unthresholded images of True1 minus Lie were subtracted from Lie minus True1. The resulting image was analyzed using MEDx 3.3 cluster detection with a minimum of z=1.645 and extent threshold of 0.05. The resulting values were used to determine local maxima and generate a visual representation of those significant clusters. The Talairach Daemon interface was used to identify location of the local maxima32. This was performed for each individual. In addition, the Talairach atlas31 was used to confirm the location of the significant clusters.

Electrodennal Activity (EDA) Analysis

In some embodiments, physiological and electrodermal activity (EDA) measures can also be obtained using techniques previously developed for polygraph systems. Hardware for gathering such measures can be used concurrently with the fMRI scan as depicted in FIG. 1A or other functional brain imaging technique.

FIGS. 1B-1F illustrate details of an exemplary skin conductance monitoring system designed to operate in a clinical magnetic resonance imaging scanner. It will monitor the electrodermal activity of a subject's skin during the acquisition of magnetic resonance images, and will filter out the electrical interference generated by the magnetic resonance imaging scanner.

The system may be easily set up in a clinical magnetic resonance imaging scanner. It immobilizes the subject's wrist and provides constant pressure on the electrodes to maintain contact against on subject's skin. It allows researchers to monitor skin conductance responses while acquiring magnetic resonance images.

FIG. 1B illustrates components of an exemplary skin conductance monitoring system. The system includes an immobilizer, a door, a shielded cable, a monitoring circuit, and a computer. The immobilizer is a glove that immobilizes the subject's wrist and holds the electrodes against the skin. The door filters out high frequency noise from the scanner but allows the low frequency skin conductance signals to pass. The shielded cable conducts signals to the electronic monitoring circuit. The electrical monitoring circuit includes a Wheatstone bridge, an instrumentation amplifier, and a Butterworth low pass filter. The computer records the skin conductance signals.

FIG. 1C illustrates details of an exemplary immobilizer. The design of the immobilizer was based on the need to 1) maintain constant pressure between the skin conductance electrodes and the subject's skin, and 2) be constructed of non-ferrous materials so as not to be pulled into the bore of the scanner's magnet. As shown in FIG. 1C, the immobilizer includes skin conductor electrode leads, twisted to reduce pickup. Plastic butterfly nuts to adjust the pressure against the hand. Foam rubber padding immobilizes the hand. Air holes provide air circulation and comfort. The cable has stress relief that is anchored to the immobilizer body so that the electrodes cannot be disturbed by movement of the subject's hand Body. The immobilizer cradles the hand and prevents movement during the experiment. AU materials may be non-ferrous, and include PVC pipe, plastic nuts and bolts, lexan sheets, and foam rubber.

FIG. 1D illustrates details of an exemplary door. The design of the door was based on the need to 1) install quickly and easily into the threshold of a clinical magnetic resonance imaging scanner room; 2) pass skin conductance signals from the interior of the room to electronic monitoring circuit outside the room; and 3) prevent radio frequency signals outside the room from traveling into the room through the signal wires.

As shown in FIG. 1D, the door includes a frame that may be made of aluminum angle braces, and is lightweight and strong. Rows of contact strips are attached to the sides and top of the door frame. This makes the door fit snugly into the threshold and electrically connects the door to the metal shielding of the scanner room. Handles allow the door to be easily lifted and moved.

A penetration panel has connectors for the twin BNC connector on the signal cable. Each line is filtered with a 400 Hz low-pass feedthrough capacitor. An inside connector attaches to connector on immobilizer cable, and an outside connector attaches to connector on shielded cable. This is shown in finer detail in FIG. 1E.

The design of the shielded cable was based on the need to pass skin conductance signals from the door to the electronic monitoring system. It may include “twinax” cable with twin BNC connectors at both ends. The entire cable may be shielded with a copper braid that is attached to the barrel of the twin BNC connectors.

FIG. 1F illustrates exemplary details of an electronic monitoring circuit. The electronic monitoring circuit includes a bridge, an amplifier, and a filter. These may be built from conventional components. However, combining all these circuits for the purpose of monitoring human skin conductance in a magnetic resonance imaging scanner is a novel combination of these elements.

Some embodiments can use electrodermal electrodes attached to the left hand and the data (sampling rate 100 per second) recorded using LabView 5.0.1 on a G4 Macintosh to gather the EDA measures. These measures can be used for a variety of purposes in conjunction with the functional brain imaging including without limitation verification, correlation and enhancement. In one embodiment, a compatible skin conductance response monitor is used such as described in copending, commonly assigned U.S. Provisional Application Ser. No. 60/341,137 (Shastri et al.), filed Dec. 13, 2001 entitled “A Skin Conductance Monitoring System for Use During Magnetic Resonance Imaging (MRI)” the content of which is hereby incorporated by this reference herein for all purposes.

The EDA data was converted to a text file by AS and AS. In order to correlate EDA with the functional BOLD signal, MEDx 3.3 analysis package requires an equal number of volumes and EDA data points. The EDA data corresponding to each volume (TR=3 seconds) was therefore averaged using STATA. Thus, every sequential 300 EDA data points (sampling rate was 100 per second) were averaged to give 272 means that corresponded to the functional brain volumes to be compared. The volumes utilized were the ones that had been motion detected, spatially normalized, smoothed, intensity normalized, and temporally filtered (see above for details). Using MEDx 3.3, independent of the deception paradigm, the changes in EDA were correlated with BOLD fMRI changes using a Pearson's r correlation. This analysis was performed for each individual resulting in a z-map. One of the correlation z-maps was found to have a significant artifact and was not included in the individual or group analysis.

For the GROUP analysis, the remaining seven individual z-maps were added using the MEDx 3.3 calculator and divided by the square root of seven. The resulting image was then analyzed with MEDx 3.3 cluster detection with a minimum of z=1.960 and spatial extent threshold of 0.05. In the direct BOLD comparison above (Lie minus True1), we were only able to use eight epochs. This study is thus underpowered relative to many in the field. For the correlational analysis, we were able to use all time points, and we were justified in using a larger z value threshold. The resulting values were used to determine local maxima and visually present the significant clusters. The Talairach Daemon interface in MEDx 3.3 was used to identify locations of the local maxima32. In addition, the Talairach atlas31 was used to confirm the location of the significant clusters and the Johns Hopkins University BRAID imaging database for Damasio Talairach space definition of orbitofrontal cortex.

For the INDIVIDUAL analysis, the individual correlation z-maps were each analyzed using MEDx 3.3 cluster detection with a minimum of z=1.960 and extent threshold of 0.05. The resulting values were used to determine local maxima and generate a visual representation of those significant clusters. The locations of the significant clusters were determined using the same technique as the group analysis.

Subjects consisted of eight healthy right-handed men (mean age 25 years with a range of 21-28) with no significant history of psychiatric or medical problems. Average Annett Handedness score for right handedness was 11 with a range of 9 to 12. All subjects correctly responded truthfully for the TRUTH room and correctly responded in the DECEPTION room. Consistency of response was monitored and all subjects reported the same object for each block as hiding the money when it did not, although the object chosen for this positive lie varied across individuals.

Group Analysis for Lie Minus True1

Image maps of the functional neuroanatomy involved in deception were generated. Within individual statistical maps to test for individual heterogeneity and the predictive power of imaging to detect deception were generated.

Lie minus True1 is the subtraction that best isolates the act of deception by controlling for most confounds. The orbitofrontal cortex (OFCx) and anterior cingulate (AC) regions of the brain were found to be significant in the deception process. The data appears in the table below:

| Z-Score | X | Y | Z | Structure |
|---|---|---|---|---|
| 3.49 | −64 | −40 | −4 | Left Middle Temporal |
| | | | | Gyrus BA21 |
| 3.05 | 56 | 12 | 8 | Right Precentral Gyrus BA |
| | | | | 44 |
| 3.00 | 44 | 44 | −8 | Right Middle Frontal Gyrus |
| | | | | (OF) |
| 2.89 | −36 | −48 | −32 | Left Cerebellum Posterior |
| | | | | Lobe |
| 2.77 | −48 | −24 | 4 | Left Superior Temporal |
| | | | | Gyrus |
| 2.73 | −56 | −56 | −8 | Left Inferior Temporal |
| | | | | Gyrus BA 37 |
| 2.48 | 20 | 56 | 12 | Right Superior Frontal |
| | | | | Gyrus |
| 2.32 | −28 | −32 | −28 | Left Cerebellum Anterior |
| | | | | Lobe |
| 2.03 | 56 | 8 | 20 | Right Inferior Frontal |
| | | | | Gyrus BA 44 (OF) |
| 2.00 | 12 | 52 | 0 | Right Anterior Cingulate |
| | | | | Cortex |

Listing Highest z score and Talairach Coordinates for Each Region
Lie - Epochs of subjects lying about the location of the money (see text)
True1 - Epochs of subjects accurately reporting location of money (see text)
BA = Brodmann Area as determined by Talairach Daemon
OF = orbitofrontal

Individual Analyses for Lie Minus True1

The heterogeneity among subjects in brain activation during the deception task was examined. Each individual was examined to determine if they had significant activation in any of these regions during the deception minus true comparison. Using a minimum statistical threshold of z=1.645 and extent threshold of 0.05, one subject had no significant activation, while seven others showed diverse activation patterns. No one brain region was found activated for all subjects when True epochs were subtracted from Lie epochs. The mean number of discrete regions identified by the group analysis that were activated by individuals was 2 per individual subject with a range of 0 to 6.

Group Analysis Correlating EDA Changes and BOLD-fMRI Changes

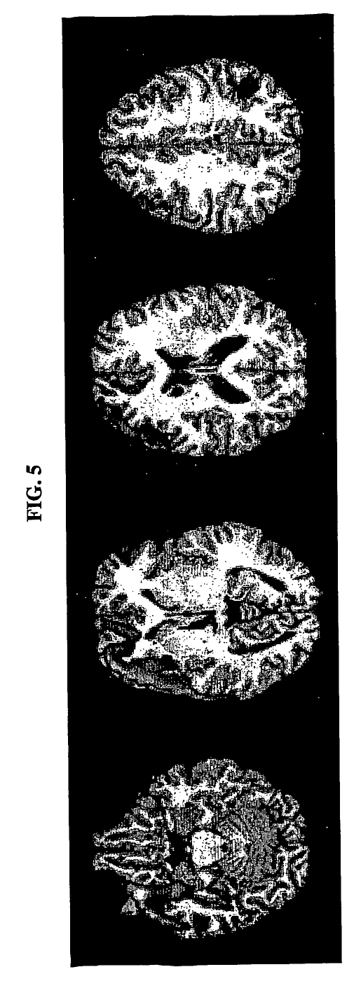

For the group analysis, one of the subjects had significant artifact after the correlational analysis and was not included in the group analysis. Significant activation was found in the orbitofrontal and right anterior cingulate gyrus as depicted in the images in FIG. 5. The analysis of this data appears in the following table.

| Z-Score | X | Y | Z | Structure |
|---|---|---|---|---|
| 11.04 | 36 | 32 | −16 | Right Inferior Frontal Gyrus (OF) |
| 6.98 | 56 | 28 | −8 | Right Inferior Frontal Gyrus GM BA |
| | | | | 47 (OF) |
| 5.11 | 56 | 32 | 16 | Right Middle Frontal Gyrus GM BA 46 |
| | | | | (OF) |
| 5.01 | 12 | 36 | 24 | Right Anterior Cingulate GM BA 32 |
| 4.27 | −48 | −48 | 40 | Left Inferior Parietal Lobule WM |
| | | | | Left Inferior Parietal Lobule GM BA |
| | | | | 40 |
| 3.89 | 12 | 8 | 12 | Right Sub-lobar Caudate GM Caudate |
| | | | | Body |
| 3.59 | 48 | 32 | 36 | Right Middle Frontal Gyrus |
| | | | | Right Middle Frontal Gyrus GM BA 9 |
| 3.51 | 64 | −32 | 4 | Right Middle Temporal Gyrus GM BA |
| | | | | 22 |
| 3.30 | 8 | −4 | −4 | Right Sub-lobar GM Hypothalamus |
| 2.73 | −4 | −24 | 40 | Left Cingulate Gyrus GM BA 31 |
| 2.63 | 56 | −40 | −16 | Right Inferior Temporal Gyrus WM |
| | | | | Right Inferior Temporal Gyrus GM |
| | | | | BA 20 |

This data demonstrates a link between EDA changes during deception and OFCx and AC activation.

Individual Analysis Correlating EDA and BOLD-fMRI Changes

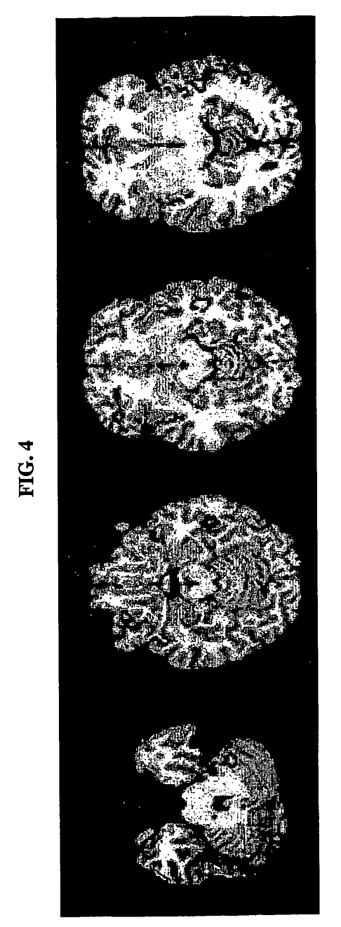

Of the seven subjects (one subject with significant artifact), six had significant (z>1.960 and extent threshold <0.05) right orbitofrontal activation (see FIG. 4), and five had significant (z>1.960 and extent threshold <0.05) right anterior cingulate activation. No other regions consistently activated across individuals.

Transcranial Magnetic Stimulation

The structural images acquired is transferred to a translational system that allows targeting specific regions in the brain based on MRI or functional brain scans. In one preferred embodiment, the translational system can be referred to as Brainsight (Rogue Research Inc.). Brainsight is an image analysis and frameless stereotaxy software system that enables the use of landmarks on the face and head (that are also identifiable on the MRI) to localize very specific areas of the brain. Other translational systems can be used within the scope of the present invention.

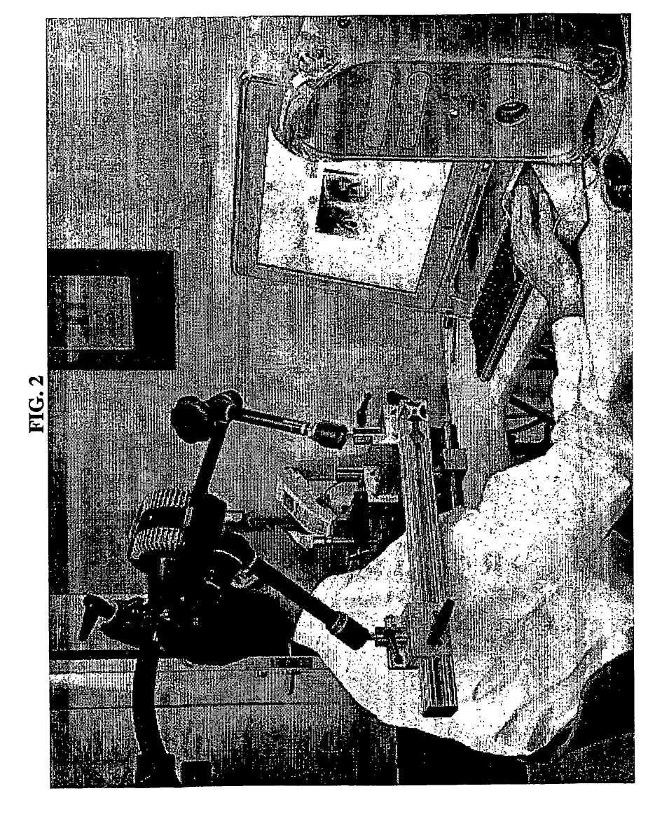

Using the fMRI analysis, the brain regions that show significant activation during deception are identified on the structural brain images. Using Brainsight, the location on the scalp over these brain regions are identified and marked. The distance from skull to cortex over the motor and prefrontal cortex is measured using Brainsight; a particular embodiment of this apparatus is depicted in FIG. 2. The TMS motor threshold is determined by using the standard method of the least percent machine output that causes the left thumb to move five out of ten times. The percent output of the TMS machine is adjusted to give 110% of the motor threshold to the prefrontal cortex; this can be accomplished in one preferred embodiment using the Bohning formula discussed below. A variety of translational systems and approaches to TMS delivery useful in the context of the present invention are discussed in copending, commonly assigned U.S. Provisional Application No. 60/367,520 (George et al.), filed Mar. 25, 2002 entitled “Methods and System of Using Transcranial Magnetic Stimulation to Enhance Cognitive Performance” and the corresponding International Application PCT/US03/09463, filed Mar. 25, 2003. The content of both of these applications is hereby incorporated by this reference herein for all purposes.

The following formula (the “Bohning formula”) can be used to adjust the dose:
Delivered intensity (% MT)=MT*(EXP(−0.36*dPC))/(EXP(−0.36*dMC))
where dPC is the measured MRI distance (in mm) from the scalp to the prefrontal cortex, and dMC is the distance for motor cortex. This formula was derived based on knowledge of TMS physics and previous measurements of TMS magnetic fields with MRI phase maps. It assumes that the effective stimulation intensity is proportional to the magnetic field measured at the center of the coil and has the same rate of exponential decrease with distance.

The frequency is set to the TMS frequencies needed to produce temporary lesions; this frequency is preferably greater than about 4 Hz. Frequencies in this range have been identified to inhibit brain functions such as language in other parts of the brain.

The TMS coil is positioned directly over the brain region identified as being activated during deception. Various coil positioning technology can be used. In one preferred embodiment, a positioning system is used such as described in copending, commonly assigned U.S. Provisional Application No. 60/381,411 (Bohning et al.), filed May 17, 2002 entitled “A TMS Coil Positioner System” and the corresponding International Application No. PCT/US03/15300 filed May 16, 2003. The content of both of these applications is hereby incorporated by this reference herein for all purposes.

While the TMS is being delivered, questions are asked and answers given by the person being interviewed. The duration of TMS firing continuously is preferably only for about 10 seconds for safety considerations. Rest periods occur between firings. These rest periods can preferably be around 20 seconds in length; however, other embodiments can use longer or shorter periods.

When the TMS is not firing, no questions are asked or answers given. Questions involve the person giving known truthful answers and possibly deceptive ones. The TMS inhibits the part of the brain that enables humans to lie. Thus, the person can only give truthful answers.

Some embodiments can include a precursor step to functional brain imaging and/or application of TMS that involves evaluating the subject for potential risk If potential risk is greater than a predetermined level with respect to a particular functional brain imaging technique, or particular parameter set associated therewith, and/or TMS configuration, or particular parameter set associated therewith, an alternative technique, configuration and/or parameter set can be used. Such an alternative technique, configuration and/or parameter set can, in certain embodiments, be subject to its own potential risk evaluation with respect to the subject.

Throughout this application, including the citation list below, various publications may have been referenced. The disclosures of these publications in their entireties are hereby incorporated by reference into this application in order to more fully describe the state of the art to which this invention pertains.

The embodiments described above are given as illustrative examples only. It will be readily appreciated by those skilled in the art that many deviations may be made from the specific embodiments disclosed in this specification without departing from the invention.

### CITATIONS

1. Amassian, V. E., Cracco, R. Q., Maccabee, P. J., Cracco, J. B., & Henry, K. (1995). Some positive effects of transcranial magnetic stimulation. Advances in Neurology, 67, 79-106.
2. Ben-Shakkar G, Dolev K (1996): Psychophysiological detection through the guilty knowledge technique: Effect of mental countermeasures. Journal of Applied Psychology 81: 273-281.
3. Bohning, D. E. (2000). Introduction and Overview of TMS Physics. In M. S. George & R. H. Belmaker (Eds.). Transcranial Magnetic Stimulation in Neuropsychiatry (pp. 1344). Washington, D.C.: American Psychiatric Press.
4. Bohning, D. E. Washington, D.C. Introduction and overview of TMS physics. In Transcranial Magnetic Stimulation in Neuropsychiatry. M. S. George and R. H. Belmaker, editors. American Psychiatric Press, 1999. 13-44.
5. Bohning, D E, Pecheny, A P, Epstein, C M, et al. Mapping transcranial magnetic stimulation (TMS) fields in vivo with MRI. NeuroReport 1997. 8:2535-2538.
6. Boroojerdi, B., Phipps, M., Kopylev, L., Wharton, C. M., Cohen, L. G., & Grafman, J. (2001). Enhancing analogic reasoning with rTMS over the left prefrontal cortex. Neurology, 56, 526-528.
7. Brasil-Neto, J. P., McShane, L. M., Fuhr, P., Hallett, M., & Cohen, L. G. (1992). Topographic mapping of the human motor cortex with magnetic stimulation: factors affecting accuracy and reproducibility. Electroencephalo Clin Neuro, 85, 9-16.
8. Brett A S, Phillips M, Beary F J III (1986): Predictive Power of the Polygraph: Can the “Lie Detector” Really Detect Liars? Lancet I (no. 8480), 544.
9. Bush G, Whalen P J, Rosen B R, Jenike M A, McInerney S C, Rauch S L (1998): The counting Stroop: an interference task specialized for functional neuroinaging—validation study with functional MRI. Human Brain Mapping. 6: 270-282.
10. Cohen, L. G., Roth, B. J., Nilsson, J., Dang, N., Panizza, M., Bandinelli, S., Friauf, W., & Hallett, M. (1990). Effects of coil design on delivery of focal magnetic stimulation. Technical considerations. Electroencephalo Clin Neuro, 75, 350-357.
11. Critchley H D, Elliot R, Mathias C J, Dolan R J (2000): Neural Activity Relating to Generation and Representation of Galvanic Skin Conductance Response: A Functional Magnetic Resonance Imaging Study. The Journal of Neuroscience 20: 3033-3040.
12. Ekman P, O'Sullivan M, Frkiesen W V, Scherer K R (1991): Invited article: Face, voice and body in detecting deceit. Journal of Nonverbal Behavior; 15: 125-135.
13. Elliot R, Rubinsztein J S, Sahakian B J, Dolan R J (2000): Selective attention to emotional stimuli in a verbal go/no-go task: an fMRI study. Neuroreport 11: 1739-1744.
14. Epstein C M, Woodard J L, Stringer A Y, Bakay A E, Henry T R, Pennell P B, Litt B: Repetitive transcranial magnetic stimulation does not replicate the WADA test. Neurology 2000, 55.
15. Farwell L A, Donchin E (1991): The Truth Will Out: Interrogative Polygraphy (“Lie Detection”) With Brain Evoked Potentials. Psychophysiology 28: 531-547.
16. Fernandez G, de Greiff A, von Oertzen J, Reuber M, Lun S, Klayer P, Ruhlmann J, Reul J, Elger C E (2001): Language mapping in less than 15 minutes: real-time functional MRI during routine clinical investigation. Neuroimage 14: 585-94.
17. Furedy J J (1986): Lie Detection as Psychophysiological Differentiation: Some Fine Lines. In: Coles M G, Donchin E, Porges S W, editors. Psychophysiology Systems, Processes and Applications. New York: The Guilford Press, pp. 683-701.
18. George M S, Ketter T A, Parekh P I, Rosinsky N, Ring H A, Pazzaglia P J, Marangell L, Post R M (1997): Blunted Left Cingulate Activation in mood Disorder Subjects During a Response Interference Task (The Stroop). Journal of Neuropsychiatry and Clinical Neurosciences 9: 55-63.
19. Honts C R, Raskin D C, Kircher J C (1987): Effects of physical countermeasures and their electromyographic detection during polygraph tests for deception. Journal of Psychophysiology; 1:21-247.
20. Kozel, F. A., Nahas, Z., DeBrux, C., Molloy, M., Lorberbaum, J. P., Bohning, D. E., Risch, S. C., & George, M. S. (2000). How the distance from coil to cortex relates to age, motor threshold and possibly the antidepressant response to repetitive transcranial magnetic stimulation. J Neuropsychiatry Clin Neurosci, 12, 376-384.
21. Kozel F A, Revell L, Lorberbaum J P, Shastri A, Nahas Z, Horner M D, Vincent D J, Lomarev M, Bohning D E, George M S: Brain Regions Involved in Deception; an fMRI study in healthy volunteers. Journal of Neuropsychiatry and Clinical Neurosciences 2001, 13; P63: p. 147. (Abstract)
22. Lancaster, J L, Rainey L H, Summerlin J L, Freitas C S, Fox P T. Automated labeling of the human brain: A preliminary report on the development and evaluation of a forward-transform method. Human Brain Mapping. 1997;5:238-242.
23. Lorderbaum J P, Newman J D, Dubno J R, Horwitz A R, Nahas Z, Teneback C C, et al. (1999): Feasibility of using fMRI to study mothers responding to infant cries. Depression and Anxiety, 10: 99-104.
24. Lubow R E, Fein 0 (1996): Pupillary size in response to a visual guilty knowledge test: New technique for the detection of deception. Journal of Experimental Psychology: Applied 2: 164-177.
25. Lykken D T (1998): A tremor in the blood: Use and abuse of the lie detector. New York: Plenum Press.
26. MacDonald A W III, Cohen J D, Stenger V A, Cartr C S (2000). Dissociating the Role of the Dorsolateral Prefrontal and Anterior Cingulate Cortex in Cognitive Control. Science 288: 1835-1838.
27. Nahas Ziad, Teneback Charlotte C, Kozel Andy, Speer Andrew M., DeBrux Cart, Molloy Monica, Stallings Lauri, Spicer Kenneth M, Arana George, Bohning Daryl E, George Mark S: Brain Effects of Transcranial Magnetic Stimulation Delivered Over Prefrontal Cortex in Depressed Adults: The Role of Stimulation Frequency and Distance from Coil to Cortex. The Journal of Neuropsychiatry and Clinical Neurosciences 2001; 13: 459470.
28. O'Doherty J, Kringelbach M L, Rolls E T, Hornak J, Andrews C (2001): Abstract reward and punishment representations in the human cortex. Nature Neuroscience 4: 95-102.
29. Pardo J V, Fox P T, Raichle M E (1991): Localization of a human system for sustained attention by positron emission tomography. Nature 349: 61-64.
30. Pavlidis I, Eberhardt N L, Levine J A (2002). Seeing through the face of deception: Thermal imaging offers a promising hands-off approach to mass security screening. Nature 415: 35.
31. Pridmore, S., Filho, J. A. F., Nahas, Z., Liberatos, C., & George, M. S. (1998). Motor threshold in Transcranial Magnetic Stimulation: A comparison of a neurophysiological and a visualization of movement method. The Journal of ECT, 14,25-27.
32. Rauch S L, Savage C R (1997): Neuropsychiatry of the Basal Ganglia: Neuroimaging and Neuropsychology of the Striatum. Psychiatric Clinics of North America 20: 741-768.
33. Roth, B. J., Saypol, J. M., Hallett, M., & Cohen, L. G. (1991). A Theoretical calculation of the electric field induced in the cortex during magnetic stimulation. Electroencephalo Clin Neuro, 81, 47-56.
34. Saypol, J. M., Roth, B. J., Cohen, L. G., & Hallett, M. (1991). A theoretical comparison of electric and magnetic stimulation of the brain. Annals of Biomedical Engineering, 19, 317-328.
35. Shastri A, Lomarev M P, Nelson S J, George M S, Holzwarth M R, Bohning D E (2001): A low-cost System for Monitoring Skin Conductance During Functional MRI. Journal of Magnetic Resonance Imaging 14: 187-193.
36. Sheehan P W, Statham D (1988): Associations between lying and hypnosis: An empirical analysis. British Journal of Experimental & Clinical Hypnosis 5: 87-94.
37. Sporer S L (1997): The less traveled road to truth: Verbal cues in deception detection in accounts of fabricated and self-experiences events. Applied Cognitive Psychology 11: 373-397.
38. Steinbrook R (1992): The polygraph test: A flawed diagnostic method. New England Journal of Medicine 327: 122-123.
39. Talairach J, Tournoux P. Co-Planar Stereotaxic Atlas of the Human Brain 3-Dimensional Proportional System: An Approach to Cerebral Imaging. New York, N.Y.: Thieme Medical Publishers, Inc.; 1988.
40. Topper, R., Mottaghy, F. M., Brugmann, M., Noth, J., & Huber, W. (1998). Facilitation of picture naming by focal transcranial magnetic stimulation of Wernicke's area. Experimental Brain Research, 121, 371-378.
41. Fowles D C, Christie M J, Edelberg R, Grings W W, Lykken D T, Venables P H. Publication Recommendations for Electrodermal Measurements, Psychophysiology:18: 232-239 (1981).
42. Technical Data Sheet “Precision Instrumentation Amplifier AD624”, Analog Devices, Norwood, Mass. (available at www.analogdevices.com).
43. Technical Data Sheet for UAF42AP, “Universal Active Filter,” Burr-Brown, Tucson, Ariz., (available at www.burr-brown.com).

What is claimed is:

1. A method for inhibiting deception, comprising the steps of:
determining at least one region of a brain used for deception; and
applying magnetic stimulation to the determined region of the brain to inhibit operation of that portion of the brain.

2. The method of claim 1, wherein the step of determining includes obtaining a functional brain map of the brain.

3. The method of claim 2, wherein the functional brain map is obtained using magnetic resonance imaging.

4. The method of claim 1, wherein the functional brain map is obtained using at least one of functional magnetic resonance imaging (fMRI), Positron Emission Tomography (PET), SPECT, qEEG, and MEG.

5. The method of claim 1, wherein the magnetic stimulation is applied to the determined portion of the brain of an individual while the individual is speaking.

6. The method of claim 5, wherein the application of the magnetic stimulation prevents the individual from telling a lie.

7. The method of claim 1, further comprising measuring physiological and electrodermal activity of the individual for verifying, correlating and/or enhancing results determined by the step of determining.

8. A system for inhibiting deception, comprising:
a device for determining at least one region of a brain used for deception; and
a magnetic stimulation device for applying magnetic stimultion to the determined region of the brain to inhibit operation of that portion of the brain.

9. The system of claim 7, wherein the determining device obtains a functional brain map of the brain.

10. The system of claim 9, wherein the functional brain map is obtained using magnetic resonance imaging.

11. The system of claim 8, wherein the functional brain map is obtained using at least one of functional magnetic resonance imageing (fMRI), Positron Emission Tomography (PET), SPECT, qEEG, and MEG.

12. The system of claim 8, whrein the magnetic stimulation is applied to the determined portion of the brain of an individual while the invidual is speaking.

13. The system of claim 12 wherein the application of the magnetic stimulation prevents the individual from telling a lie.

14. The system of claim 8, further comprising a device for measuring physiological and electrodermal activity for verifying, correlating and/or enhancing results determined by the determining device.
